(19) United States
(12) Patent Application Publication
(10) Pub. No.: US 2026/0102537 A1
(43) Pub. Date: Apr. 16, 2026

(54) TINTED FILLER AND METHOD

(71) Applicant: Prollenium Medical Technologies, Inc., Richmond Hill (CA)

(72) Inventors: Hitoshi Masui, Richmond Hill (CA); Timothy Lee, Richmond Hill (CA); Ario Khoshbin, Richmond Hill (CA)

(73) Assignee: Prollenium Medical Technologies, Inc., Richmond Hill (CA)

(21) Appl. No.: 19/360,336

(22) Filed: Oct. 16, 2025

Related U.S. Application Data
(60) Provisional application No. 63/707,930, filed on Oct. 16, 2024.

(51) Int. Cl. A61L 27/20; A61L 27/54

(52) U.S. Cl. CPC A61L 27/20; A61L 27/54

(57) ABSTRACT

A dermal filler composition including a coloring agent dispersed in a continuous phase composed of cross-linked hyaluronic acid gel particles, wherein the coloring agent is one or more of vitamin B2 and B12.

### CROSS-REFERENCE TO RELATED APPLICATIONS

The present disclosure claims the benefit of U.S. Provisional Patent Application No. 63/707,930, filed Oct. 16, 2024, the entirety of which is incorporated by reference herein.

### FIELD OF THE INVENTION

The invention relates to the field of dermal fillers. More particularly, the invention relates to hyaluronic acid compositions used as dermal fillers and a method of manufacture of hyaluronic acid compositions.

### BACKGROUND OF THE INVENTION

Hyaluronic acid (HA) is a linear macromolecular polysaccharide consisting of alternately bonded 3-D-N-acetylglucosamine and 3-D-glucuronic acid. It is well known to inject hyaluronic acid under the dermis for cosmetic reasons. Such dermal fillers can be used to offset the effects of aging on the skin, by smoothing soft tissue defects like nasolabial folds and marionette lines as well as more substantial augmentation such as smoothing hollow cheeks resulting from lipoatrophy or enhancing the fullness of lips. Fillers must be able to satisfy a number of needs depending on the type of defect which needs to be corrected. In some embodiments, the dermal fillers can be compositions of HA including additional components for aesthetic reasons.

### SUMMARY OF THE INVENTION

Forming one aspect of the invention is a dermal filler composition including a coloring agent dispersed in a continuous phase composed of cross-linked hyaluronic acid gel particles. The coloring agent is one or more of vitamin B2 and B12.

The compositions may be used for face augmentation procedures.

### DETAILED DESCRIPTION

One embodiment of the invention is a dermal filler composition comprising: a coloring agent dispersed in a continuous phase composed of cross-linked hyaluronic acid gel particles, wherein the coloring agent is vitamin B2 at a concentration of 1 mg per ml. The hyaluronic acid gel is REVANESSE ULTRA®.

One embodiment of the invention is a dermal filler composition comprising: a coloring agent dispersed in a continuous phase composed of cross-linked hyaluronic acid gel particles, wherein the coloring agent is vitamin B12 at a concentration of 1 mg per ml. The hyaluronic acid gel is REVANESSE ULTRA®.

U.S. patent application Ser. No. 16/592,151 , filed Oct. 3, 2019, is incorporated herein by reference.

Whereas specific embodiments are herein shown and described, variations are possible.

1. A dermal filler composition comprising: a coloring agent dispersed in a continuous phase composed of cross-linked hyaluronic acid gel particles, wherein the coloring agent is one or more of vitamin B2 and B12.
